(19) United States
(12) Patent Application Publication
(10) Pub. No.: US 2011/0152766 A1
(43) Pub. Date: Jun. 23, 2011

(54) IMPLANT WITH COATING

(76) Inventors: Andreas Hofmann, Graefenberg (DE); Alexander Borck, Aurachtal (DE); Michael Tittelbach, Nuernberg (DE)

(21) Appl. No.: 12/961,931

(22) Filed: Dec. 7, 2010

Related U.S. Application Data
(60) Provisional application No. 61/288,347, filed on Dec. 21, 2009.

(51) Int. Cl. A61K 31/337; A61F 2/00; A61P 29/00; A61P 35/00; A61P 21/02; A61M 25/10; B05D 3/10

(52) U.S. Cl. 604/103.02; 424/423; 514/449; 427/2.3

(57) ABSTRACT

One embodiment of the invention relates to an implant with a coating containing at least one active pharmaceutical substance. The inventive implant is characterized in that the coating is covered by a protective layer comprising or containing one or more materials from the group including shellac, vinylpyrrolidone-vinyl acetate copolymer, vinyl acetate-crotonic acid copolymer, vinyl acetate-vinyl propionate-crotonic acid terpolymer, methylvinyl ether-maleic anhydride copolymer, vinylpyrrolidone-dimethylaminoethyl acrylate copolymer, polyvinylpyrrolidone, polyvinyl acetate, polycrotonic acid, polyvinyl propionate, polymethylvinyl ether, polymaleic anhydride and polydimethylaminoethyl acrylate.

### CROSS REFERENCE

The present application claims priority on U.S. Provisional Patent Ser. No. 61/288,347 filed Dec. 21, 2009; which application is incorporated by reference herein.

### TECHNICAL FIELD

One example embodiment of the invention relates to a medical implant having a coating comprising at least one active pharmaceutical substance.

### BACKGROUND

Implants such as stents or catheters are tubes of different diameters, which may be inserted into the respective body cavity to be treated. Then a tube having a folded undilated balloon in a predefined region of the tube is advanced along the guide wire up to the location in the vessel to be treated, so the balloon is placed in the area of the vessel to be treated, e.g., a stenosis. Then the balloon is dilated, i.e., unfolded and expanded, so the site to be treated is reopened or widened and the flow of body fluid in the vessel is not hindered or is no longer hindered to the previous extent. Finally, the balloon is emptied again and removed from the vessel along the guide wire. Simultaneously or subsequently, the guide wire is also retracted out of the vessel.

In recent years, various medication-coated stents and balloon catheters have been developed and approved for treatment of coronary and peripheral vascular diseases in humans. The medication coating is applied to the surface of the stent or balloon catheter. The catheter is advanced up to the lesion over a guide wire and through a guide catheter. During this procedure, the catheter is exposed to various mechanical influences, which can lead to abrasion and loss of the coating. These include, among others, removal of the protector, contact with the balloon by the surgeon, kinking of the balloon, pushing the catheter through the so-called introducer or through a shunt, friction in the guide catheter, contact with blood, friction on the vascular wall, which presents a high mechanical resistance, especially in the case of calcified lesions.

The loss of pharmacologically active substance en route to the destination site necessitates a much higher active substance loading of the stent or balloon catheter than is actually necessary therapeutically. This can be undesirable.

Two goals must thus be achieved with the active substance coating: effective and reproducible transport of the active substance up to the target site and rapid release of a sufficient dose to the vascular wall at the moment of dilatation of the stent and/or balloon. However, the interactions between the stent surface or balloon surface and the active substance are limited. In addition, many active substances in general tend to form crystallites or solid phases, which are fragile and tend to disintegrate due to mechanical loading. In the past, medication-coated implants have been manufactured, so that the coating is stable but dispenses only a relatively small amount of substance to the lesion, or so that the coating is mechanically fragile and there is a loss of active substance before administration at the site of the lesion.

Approved medication-coated stents or balloon catheters for treatment of coronary and peripheral vascular diseases in humans are based in particular on the use of the active substance paclitaxel. Paclitaxel is a strongly hydrophobic molecule, which tends to form crystalline structures. The crystallinity of the substance limits its bioavailability. The crystal lattice must be broken open in order for the active substance to be solvated and thus be available. Furthermore, parts of the active substance coating are released due to frictional forces of the balloon in the guide catheter and in the vascular system, if the mechanical stability of the coating is too low.

### SUMMARY

One embodiment of the invention provides an approach, which will reduce the active substance loading of the implant while at the same time not reducing the availability of the active substance at the application site, but with far fewer losses occurring on the path to the application site. Due to the lower dosage, the total burden for the patient is reduced and the reproducibility of the dose that has an actual therapeutic effect is increased.

Some embodiments of the invention comprise an implant with a coating comprising at least one active pharmaceutical substance. In some embodiments, two, three or more active pharmaceutical substances are present in combination. Other components may also be present. The example inventive implant is characterized in that the coating is covered by a protective layer, comprising or containing one or more materials from the group comprising vinyl pyrrolidone-vinyl acetate copolymer, vinyl acetate-crotonic acid copolymer, vinyl acetate-vinyl propionate-crotonic acid terpolymer, methylvinyl ether-maleic anhydride copolymer, vinylpyrrolidone-dimethylaminoethyl acrylate copolymer, polyvinyl pyrrolidone, polyvinyl acetate, polycrotonic acid, polyvinyl propionate, polymethylvinyl ether, polymaleic anhydride and polydimethylaminoethyl acrylate.

### DETAILED DESCRIPTION

An invention embodiment is based on the discovery of applying a temporary protective layer to the active substance-laden coating to thereby protect this active coating from mechanical stress. In other words, the coating containing the active substance is affixed temporarily by applying a protective layer. Through this fixation, most of the medication reaches the intended site. The chemical nature of the polymers used nevertheless makes it possible for the active substance to be delivered quantitatively at the intended site. The polymers that are used in at least some embodiments are water-soluble, but the dissolving process, which is controlled by the molecular weight, is not spontaneous and instead takes place with a delay including a swelling phase, which may last between one and ten minutes when in a physiological environment. Other dissolution periods may be useful in other invention embodiments as discussed below. During this period of time, the medication is protected by the polymer film on the balloon catheter. After dilatation, the completely swollen film together with the active substance is pressed against the vascular wall and can then manifest its effect after transfection. Thus, on the whole, a more homogeneous distribution of the active substance at the lesion is achieved, the loss of active substance en route to the lesion is reduced (since it is protected by the polymer film protective layer en route to the lesion), leading to a lower systemic burden for the patient. Thus the protective layer forms a closed shell around the medication coating to thereby encapsulate the coating and protect it from friction, other mechanical interaction, chemical interaction, and exposure to the surrounding environment until the protective layer is dissolved to expose the underlying coating.

The distribution of the active substance is more homogenous than in the prior art since it avoids mechanical interaction during placement of the implant and thereby remains distributed as desired on the implant until the protective layer dissolves. Certainty of delivery of active substance is also improved over the prior art since greater certainty is made regarding the location the implant will be when the active substance coating is exposed.

The thicknesses of the active substance coating and the protective layer may be varied as may be desirable for particular applications. For example, the desired active substance loading and concentration may determine the thickness of the active substance coating. The thickness of the protective layer may be varied as desired, for example, to control the timing of dissolution. Generally, thicker layers will require more time for dissolution than thinner layers. Composition of the protective layer may also be useful to control dissolution timing. Timing of dissolution may thereby be controlled using factors including layer thickness and composition. In some different invention embodiments the layer may be configured to remain in place for at least about 1 minute, at least about 5 minutes, at least about 10 minutes, and at least about 30 minutes, when in a physiological environment. As used herein, the term “physiological environment” is intended to broadly refer to an environment such as the human body that a stent or catheter will encounter during use in a human. The dissolution rate may also be controlled by varying the molecular weight of the polymer composition of the protective layer.

The term “active pharmaceutical substance” (or active therapeutic substance or active substance) in the sense of the present invention is understood to be an active substance (medication) of animal, plant or synthetic origin, which is used in a suitable dosage as a therapeutic agent to influence states or functions of the body, as a substitute for natural active substances produced by the human or animal body and to eliminate or render harmless disease pathogens, tumors, cancer cells or exogenous substances.

Such active pharmaceutical substances have, for example, an anti-inflammatory and/or antiproliferative and/or spasmolytic effect, so it is possible to avoid restenoses, inflammations or (vascular) spasms, for example. The active pharmaceutical substance may be, for example, one or more of an anti-inflammatory, and antiproliferative and an spasmolytic. Such substances may comprise in particular one or more substances from the group of active substances including calcium channel blockers, lipid regulators (for example, fibrates), immunosuppressants, calcineurin inhibitors (for example, tacrolimus), antiphlogistics (for example, cortisone or diclofenac), anti-inflammatories (for example, imidazoles), anti-allergies, oligonucleotides (for example, dODN), estrogens (for example, genistein), endothelializers (for example, fibrin), steroids, proteins, hormones, insulins, cytostatics, peptides, vasodilators (for example, sartans) and substances having an antiproliferative effect, namely taxols or taxans, in this case preferably paclitaxel.

The active pharmaceutical substance is present in the coating of at least some invention embodiments either in pure form or together with other pharmaceutical excipients. Suitable excipients include, for example, benzalkonium chloride, α-tocopherol, glucose, lactose, calcium phosphate, calcium biphosphate, sodium bicarbonate, sodium carbonate, titanium oxide, zinc oxide, magnesium oxide, silicates such as highly dispersed silicon dioxide (colloidal silicic acid, Aerosil, SiO₂), talc, kaolin, bentonite, aliphatic alcohols, DMSO, glycerol, propylene glycol, stearic acid, sugars and sugar alcohols, lactose, cyclodextrins such as α-cyclodextrin, β-cyclodextrin, γ-cyclodextrin, mannitol, sorbitol, starches, cellulose powders, cellulose esters and ethers, such as methylcellulose, ethylcellulose, hydroxyethyl-cellulose, hydroxypropylcellulose, hydroxypropylmethylcellulose, sodium carboxymethyl-cellulose, cellulose acetate phthalate, hydroxypropylmethylcellulose acetate phthalate and microcrystalline cellulose, gelatins, squalene and other isoprene units, gum arabic, pectin, xanthan, alginate, shellac, polyacrylic acids, such as carbomers, polyvinylpyridine, polyvinyl pyrrolidone polyvinyl alcohol, polyvinyl acetate and mixtures of polyvinylpyridine and polyvinyl acetate, polyethylene glycol, liquid petrolatum, synthetic and natural fats and silicones, amphiphilic or surfactant excipients, such as anionic surfactants, saponides; cationic surfactants, such as cetylpyridinium chloride, nonionic surfactants; polyoxyethylene sorbitan, macrogol glycerol fatty acid esters, fatty alcohol ethers of polyoxyethylene, fatty acid esters of sucrose and especially D-α-tocopheryl-1000-succinate, amphoteric surfactants, complex emulsifiers such as cetylstearyl alcohol (types A and B), quaternary ammonium compounds and preservatives, and antioxidants such as citric acid, citraconic acid, tartaric acid, mono- and polyphosphates, organic phosphates such as dodecyl phosphate, hexose phosphate and hyaluronidases or hyaluronate lyases, butyryl-n-trihexyl citrate (BTHC) and triethyl citrate.

In some embodiments, the material is preferably a vinyl pyrrolidone-vinyl acetate copolymer, in particular a vinyl pyrrolidone-vinyl acetate copolymer, in which the ratio of pyrrolidone radicals to acetate radicals is between about 15:85 to 55:45. Other ratios may also be useful. The specified ratio range, however, is believed to provide particular benefits and advantages in many applications. Copolymers of the aforementioned composition have a hardness and solubility that are especially suitable for many applications.

Another aspect of embodiments of the present invention lies in the use of a stent or balloon catheter designed for the preceding embodiments accordingly for treatment of coronary and peripheral vascular diseases in humans.

Traditional elements of stents, catheters and other implants are generally known and for sake of brevity need not be discussed in detail herein. They may be made of biocompatible materials, including magnesium alloys as well as other metallic and non-metal materials. The implant includes at least an outer surface on which the coating including at least one pharmaceutically active surface is applied, which is then overlaid with the protective layer.

Further aspects of invention embodiments may be appreciated by considering the following example embodiments.

An example embodiment of the invention is production of a paclitaxel-coated stent or balloon catheter by a method comprising the steps of:

1. Coating the stent or catheter with the active substance
a. with paclitaxel as a pure active substance or
b. with paclitaxel in a formulation together with a pharmacological excipient.

2. Aftertreatment with a film-forming polymer for short-term fixation of the active substance layer with a protective layer.

The protective layer thus forms a closed shell around the medication coating to encapsulate it until the shell dissolves, and can be applied by suitable methods. Many such methods are known and will be suitable, with some including application by spraying, drops, immersion, condensation, atomization or vaporization.

Suitable excipients may be selected from the list given above, for example. Others will also be suitable.

The protective layer of the additional materials mentioned above may be applied by a similar method, including for example application by spraying, drops, immersion, condensation, atomization or vaporization.

Embodiments of the invention further include stents, catheters and other implants made through methods of the invention. Still other invention embodiments include methods for using such stents, catheters and implants.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. An implant with a coating comprising at least one active pharmaceutical substance, characterized in that the coating is covered by a protective layer comprising one or more materials from the group including shellac, vinylpyrrolidone-vinyl acetate copolymer, vinyl acetate-crotonic acid copolymer, vinyl acetate-vinyl propionate-crotonic acid terpolymer, methylvinyl ether-maleic anhydride copolymer, vinylpyrrolidone-dimethylaminoethyl acrylate copolymer, polyvinylpyrrolidone, polyvinyl acetate, polycrotonic acid, polyvinyl propionate, polymethylvinyl ether, polymaleic anhydride and polydimethylaminoethyl acrylate.

2. The implant according to claim 1, wherein the material is a vinylpyrrolidone-vinyl acetate copolymer.

3. The implant according to claim 2, wherein the ratio of pyrrolidone radicals to acetate radicals of the vinylpyrrolidone-vinyl acetate copolymer is between 15:85 to 55:45.

4. The implant according to claim 1, wherein the active pharmaceutical substance is paclitaxel.

5. The implant according to claim 1, wherein the implant is a stent or balloon catheter.

6. The use of the implant according to claim 1, for treatment of coronary and peripheral vascular diseases in humans.

7. The implant according to claim 1 wherein the implant is comprised of a magnesium alloy.

8. The implant according to claim 1 wherein the pharmaceutically active material is one or more of an anti-inflammatory, and antiproliferative and an spasmolytic.

9. The implant according to claim 1 wherein the protective layer is temporary and completely encapsulates the underlying coating.

10. The implant according to claim 1 wherein the protective layer is configured to dissolve in a period of between about 1 to about 10 minutes when in a physiological environment, whereby the coating is encapsulated when the implant is in such an environment for a period of between about 1 and about 10 minutes and is exposed thereafter.

11. The implant according to claim 1 wherein the protective layer is configured to dissolve in a period of between about 1 to about 10 minutes when in a physiological environment, whereby the coating is covered and protected from exposure when the implant is used in such an environment for a period of at least about 10 minutes and is exposed after the protective layer has dissolved.

12. A surgical implant comprising:
a base body having a surface;
a coating applied to the surface comprising at least one pharmaceutically active material; and
a temporary polymer film protective layer over the coating, the protective layer comprising one or more water soluble polymers that form a hard shell that dissolves in a period of time of between about one and ten minutes in a physiological environment, wherein the temporary protective layer encapsulates the underlying coating for a period of at least about one minute in a physiological environment until it is dissolved to expose the coating.

13. A surgical implant as defined by claim 12 wherein the temporary protective layer dissolves with a swelling phase.

14. A surgical implant as defined by claim 12 wherein the temporary protective layer comprises a vinylpyrrolidone-vinyl acetate copolymer.

15. A surgical implant as defined by claim 12 wherein the protective layer comprises one or more materials from the group including shellac, vinylpyrrolidone-vinyl acetate copolymer, vinyl acetate-crotonic acid copolymer, vinyl acetate-vinyl propionate-crotonic acid terpolymer, methylvinyl ether-maleic anhydride copolymer, vinylpyrrolidone-dimethylaminoethyl acrylate copolymer, polyvinylpyrrolidone, polyvinyl acetate, polycrotonic acid, polyvinyl propionate, polymethylvinyl ether, polymaleic anhydride and polydimethylaminoethyl acrylate.

16. A surgical implant as defined by claim 12, wherein:
the water soluble polymers comprise a vinylpyrrolidone-vinyl acetate copolymer having a ratio of pyrrolidone radicals to acetate radicals between 15:85 to 55:45; and
the pharmaceutically active material comprises paclitaxel.

17. A method for making a surgical implant comprising the steps of:
applying a coating to the implant that includes a pharmaceutically active material;
encapsulating the coating with a temporary protective layer for short term fixation of the coating, the temporary layer forming a hard film made using one or more water soluble polymers, the protective layer configured to dissolve in a period of more than 1 minute when exposed to a physiologic environment to thereby expose the coating.

18. A method for making a surgical implant as defined by claim 17 wherein:
the coating comprises one or more of a an anti-inflammatory, and antiproliferative and an spasmolytic; and,
the coating is made using a vinylpyrrolidone-vinyl acetate copolymer and is configured to dissolve in a period of between about 1 and 10 minutes when exposed to a physiologic environment.

19. A method for making a surgical implant as defined by claim 17 wherein the coating comprises paclitaxel in a formulation with at least one pharmacological excipient.
